(19) United States
(12) Patent Application Publication
(10) Pub. No.: US 2007/0005079 A1
(43) Pub. Date: Jan. 4, 2007

(54) System, apparatus, and method for repairing septal defects

(76) Inventors: David Zarbatany, Laguna Niguel, CA (US); Tai Tieu, Fountain Valley, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/174,143

(22) Filed: Jun. 30, 2005

(51) Int. Cl. A61B 17/10

(52) U.S. Cl. 606/139

(57) ABSTRACT

An apparatus and method for repairing septal defects includes advancing a catheter to the site of the defect, grasping opposing edges of the defect, passing one or more suture lines through the opposing edges, and tightening the suture lines. The catheter can include one or more vacuum ports, with the vacuum ports being sized and configured to grasp opposing edges of the defect when vacuum is applied thereto. The vacuum ports may be positioned longitudinally distant from each other along the catheter, and may also be on different sides of the catheter. The vacuum ports may have vacuum applied via independent control. The catheter can also include suture deploying structure that prevents the suture line from becoming entangled in the catheter.

### FIELD OF THE INVENTION

The present invention relates to medical devices and methods. In particular, the present invention relates to a system, apparatus, and method for repairing tissue, and particularly for repairing septal defects, such as a patent foramen ovale (PFO).

### BACKGROUND OF THE INVENTION

Septal defects are a relatively common occurrence. While many septal defects are relatively benign and have little or no impact on a person's health, other septal defects can be more serious.

One type of septal defect is a patent foramen ovale (PFO), which is an opening between the right atrium and the left atrium. Because the fetal lungs do not provide air prior to birth, fetal blood is oxygenated by the mother via the umbilical cord and placentia. To provide for such circulation, the fetal blood circulation system includes several vessels and openings that remain open during fetal development but that close soon after birth. One such opening is the foramen ovale, which permits blood to flow from the right atrium into the left atrium in a fetal heart, thereby allowing blood to bypass the fetal lungs and flow directly from the venous circulation to the arterial circulation.

After birth, the infant's lungs typically provide oxygenation to the blood, and it is generally undesirable to continue having blood flow from the venous circulation to the arterial circulation without first passing through the lungs. Accordingly, it is generally desirable that the foramen ovale be closed after birth.

At birth, left atrial pressure increases as the pulmonary circulation is established. For most newborn infants, this pressure increase causes the closure of a flap of tissue which occludes the foramen ovale and then heals in the occluded position shortly after birth. In a significant percentage of persons, however, the tissue flap does not heal to permanently occlude the foreman ovale. This condition is known as a patent (i.e., open) foramen ovale (PFO).

While a PFO can be a relatively benign condition, PFOs have been associated with migraines. PFOs can also cause strokes by permitting blood containing small thrombi to bypass the lungs (which would otherwise filter out such small thrombi) and flow directly from the venous circulation to the arterial circulation and into the brain.

Treatments for PFOs range from open-heart surgery to percutaneous procedures. Open-heart surgery for PFOs typically involves suturing the PFO closed. Although relatively simple, such open-heart surgical treatment is associated with all the usual risks of cardiac surgery. Percutaneous methods include deploying mesh, clamshell, or other similar implanted devices to close the PFO. Other treatments include using heat, laser, RF, or other energy to treat the tissue of (or adjacent to) the PFO to induce the tissue to permanently close the PFO. The percutaneous methods are often complicated and may involve relatively large implanted devices or uncertain tissue treatments.

In light of the foregoing, there is presently a need for improved systems for treating PFOs. More specifically, there is a present need for an improved method, apparatus, and system for repairing PFOs. The current invention meets this need.

### BRIEF SUMMARY OF THE INVENTION

The present invention solves the problem of effectively treating an opening tissue, such as a PFO or other septal defect. Additionally, the present invention provides a device capable of treating a PFO via a catheter from a remote insertion location.

In one aspect, the present invention is directed to a system for repairing a PFO and includes a treatment catheter having at least one vacuum recess and capable of applying at least one suture to the tissue adjacent the PFO.

The invention can further have a fastener catheter capable of attaching at least one fastener to the suture. In addition, the fastener catheter can include at least one cutting member configured to cut the suture to a desired length.

In another aspect, the present invention pertains to a device for repairing a PFO including a treatment catheter having at least one needle lumen in communication with at least one needle port positioned therein, and at least one needle positioned within the needle lumen.

In yet another aspect, the present invention discloses a system for repairing tissue within the heart of a patient and includes a guide wire capable of being inserted into the patient and advanced through a circulatory pathway, a treatment catheter attachable to the guide wire and capable of applying at least one suture to the tissue, and a fastener catheter attachable to the guide wire and capable of attaching at least one fastener to the suture.

In another aspect, the present invention discloses a catheter for delivering a suture to tissue within the heart of a patient and includes an elongated body having a distal end, at least one suction recess formed on the distal end, at least one needle port located proximate to the suction recess, at least one needle lumen having at least one needle positioned therein in communication with the needle port, at least one needle receiving port having at least one needle catch located therein positioned proximate to the suction recess, and at least one actuator member in communication with the needle.

In yet another aspect, the present invention is directed to a catheter for delivering a suture to septal tissue within the heart of a patient and comprises an elongated body having a distal end with at least first and second suction recesses formed thereon, with the first and second suction recesses circumferentially displaced about the elongated body. In a further aspect, the first and second suction recesses may also be longitudinally displaced along the elongate body.

In another aspect, the present invention involves a device for applying suture to tissue, such as tissue adjacent a PFO, and includes a catheter body having a proximal end and a distal end, at least one suction recess adjacent the distal end, at least one needle port located proximate to or within the suction recess, at least one needle lumen having at least one detachable needle attached to suture material positioned therein and in communication with the needle port, at least one needle receiving port located proximate to the suction recess, at least one needle trap capable of receiving the detachable needle positioned within the needle receiving port, and at least one actuator member in communication with the needle.

In another aspect, the present invention involves a device for applying suture to tissue, such as tissue adjacent a PFO, and includes a catheter body having a proximal end and a distal end, a first and a second suction recess adjacent the distal end, first and second needle ports located respectively proximate to or within the first and second suction recesses, each needle port having at least one needle lumen having at least one advanceable and retractable needle therein, at least one needle receiving port located proximate to or within the suction recess, each needle receiving port further having at least one needle catcher attached to suture material positioned therein and in alignment to receive a needle in the advanced position, and at least one actuator member in communication with the needle.

The present invention also discloses various methods of treating a PFO within the body of a patient. In one aspect, a method of treating a PFO is disclosed which includes advancing a guide catheter through a circulatory pathway to a location in the heart proximate to a PFO, advancing a PFO treatment catheter through the guide catheter to the PFO, applying a vacuum to stabilizing a first adjacent tissue portion with the treatment catheter, deploying a first suture into the stabilized first adjacent tissue portion, applying a vacuum to stabilize a second adjacent tissue portion with the treatment catheter, deploying a second suture into the second adjacent tissue portion, removing the vacuum to disengage the first and second adjacent tissue portions from the treatment catheter, and joining the first and second adjacent tissue portions by reducing the distance between the first and second sutures.

An alternate method of treating a PFO is disclosed and comprises advancing a guide catheter through a circulatory pathway to a location in the heart proximate the PFO, advancing a PFO treatment catheter through the guide catheter to the PFO, applying a vacuum to stabilizing a first adjacent tissue portion with the treatment catheter, deploying a first suture into the stabilized first adjacent tissue portion, applying a vacuum to stabilize a second adjacent tissue portion with the treatment catheter, deploying a second suture into the second adjacent tissue portion, removing the vacuum to disengage the first and second adjacent tissue portions from the treatment catheter, and removing the therapy catheter from the guide catheter. A fastener catheter is positioned over the first and second suture and advanced through the guide catheter to the heart valve. Once positioned, the first and second leaflets are joined by reducing the distance between the first and second sutures and a fastener is deployed from the fastener catheter.

Other objects, features, and advantages of the present invention will become apparent from a consideration of the following detailed description.

### BRIEF DESCRIPTION OF THE DRAWINGS

FIG. 1 illustrates a treatment catheter advanced within a patient's vasculature and into a patent foramen ovale according to an embodiment of the invention;

FIG. 2 is a perspective view of a treatment catheter according to an embodiment of the invention;

FIG. 3 is a side view of a treatment catheter according to an embodiment of the invention;

FIG. 4 is a perspective view of a distal end of a treatment catheter according to an embodiment of the invention;

FIG. 5 is a perspective view of a proximal end of a treatment catheter according to an embodiment of the invention;

FIG. 6 is a side view of a distal end of a treatment catheter positioned within a PFO according to an embodiment of the invention;

FIG. 7 is a side view of a distal end of a treatment catheter positioned within a PFO according to an embodiment of the invention;

FIG. 8 is a side view of a distal end of a treatment catheter positioned within a PFO according to an embodiment of the invention;

FIG. 9 is a side view of a distal end of a treatment catheter positioned within a PFO according to an embodiment of the invention;

FIG. 10 is a side view of a distal end of a treatment catheter positioned within a PFO according to an embodiment of the invention;

FIG. 11 illustrates a treatment catheter being removed from a patient's vasculature during treatment of a PFO according to an embodiment of the invention;

FIG. 12 is a side view of a distal end of a treatment catheter according to an embodiment of the invention;

FIG. 13 is a side view of a distal end of the treatment catheter of FIG. 12 positioned within a PFO according to an embodiment of the invention;

FIG. 14a shows a perspective view of an embodiment of the fastener catheter of the present invention;

FIG. 14b shows a perspective view of an embodiment of the fastener catheter handle of the present invention;

FIGS. 15a and 15b show a perspective view of the components of the fastener tip of the present invention;

FIG. 15c shows a perspective view of the fastener tip of the present invention having a fastener attached thereto;

FIG. 16a shows a side view of an embodiment of the fastener of the present invention;

FIG. 16b shows a side view of the fastener of the present invention securing suture passed through tissue;

FIG. 17 illustrates a fastener catheter advanced within a patient's vasculature and adjacent a patent foramen ovale according to an embodiment of the invention;

FIG. 18 illustrates a suture and clip in place to secure a foramen ovale after treatment according to an embodiment of the invention;

FIG. 19 illustrates a suture and knot in place to secure a foramen ovale after treatment according to an embodiment of the invention;

FIG. 20 is a side view in cross-section of a treatment catheter according to an embodiment of the invention; and

FIG. 21 depicts illustrates a guide catheter advanced within a patient's vasculature and adjacent a patent foramen ovale according to an embodiment of the invention

### DETAILED DESCRIPTION OF THE INVENTION

The invention is an apparatus, system, and method for treating a patent foramen ovale (PFO) to cause closure thereof. More specifically, the invention provides for percutaneous or other minimally-invasive application of suture to PFO to cause closure of the PFO.

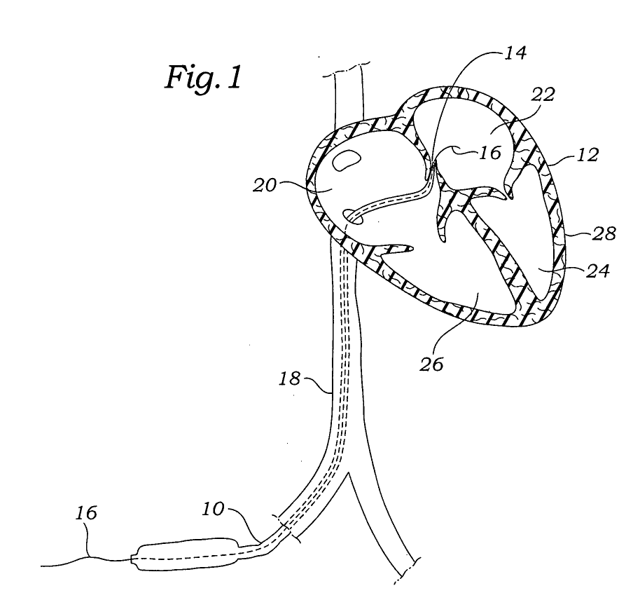

FIG. 1 depicts a catheter 10 according to the invention being advanced through a patient's vasculature to a heart 12 and into a PFO 14. A guidewire 16 has previously been advanced through the vasculature by passing up the inferior vena cava 18, through the right atrium 20, and through the PFO 14 and into the left atrium 22. Note that other introductory routes, including other percutaneous and minimally invasive routes, are also within the scope of the invention. For example, the guidewire and device could be introduced through heart vessels leading to the left ventricle 24 or right ventricle 26, and then on to access the PFO 14 through either the right atrium 20 or left atrium 22. Depending on the particular embodiment, the device may also be introduced through the heart wall 28, as may be the case in a minimally-invasive surgical procedure conducted through a patient's chest cavity. The particular route selected for introduction of the device to the PFO 14 depends on various factors, including the condition of the patient. While the embodiment of FIG. 1 includes a guidewire 16 to guide the treatment catheter 10 into position, the guidewire 16 does not have to be present in all embodiments, such as where the treatment catheter 10 is steerable on its own to the PFO 14.

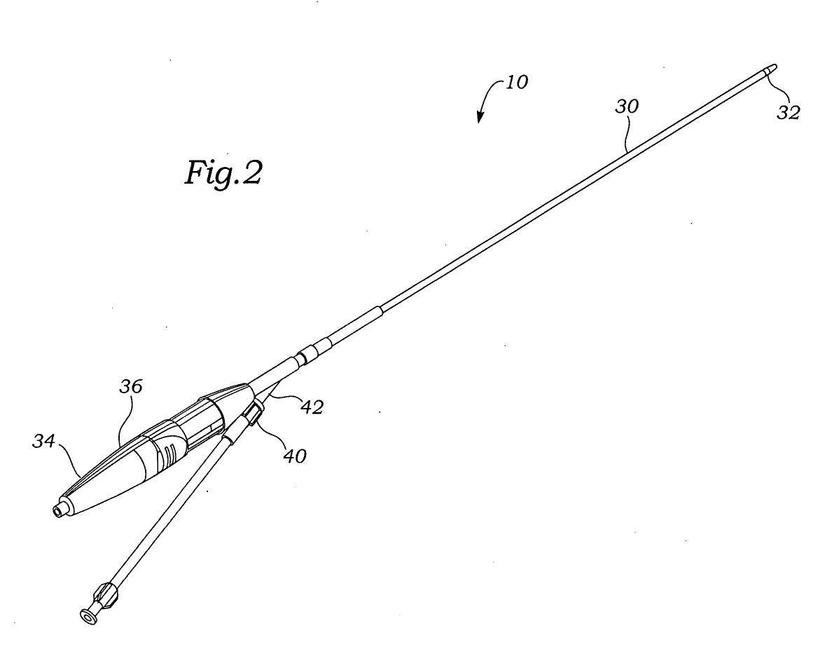

FIG. 2 depicts another view of the treatment catheter 10. The treatment catheter 10 comprises a generally elongated body 30 having a distal end 32 and a proximal end 34. A handle 36 is located at the proximal end 34. The treatment catheter 10 has sufficient length to reach the PFO 14 from outside the patient's body via the particular route selected. For a percutaneous route, the treatment catheter will generally have a length on the order of 60 to 75 cm. Other access routes may require different lengths. The elongated body 30 and distal end 32 have a diameter that is small enough to pass through the particular blood vessels and/or openings of the particular access route selected. While percutaneous approaches through the inferior vena cava, as depicted in FIG. 1, can accommodate diameters of 12 to 16 Fr, other approaches may accommodate and/or require smaller or larger diameters.

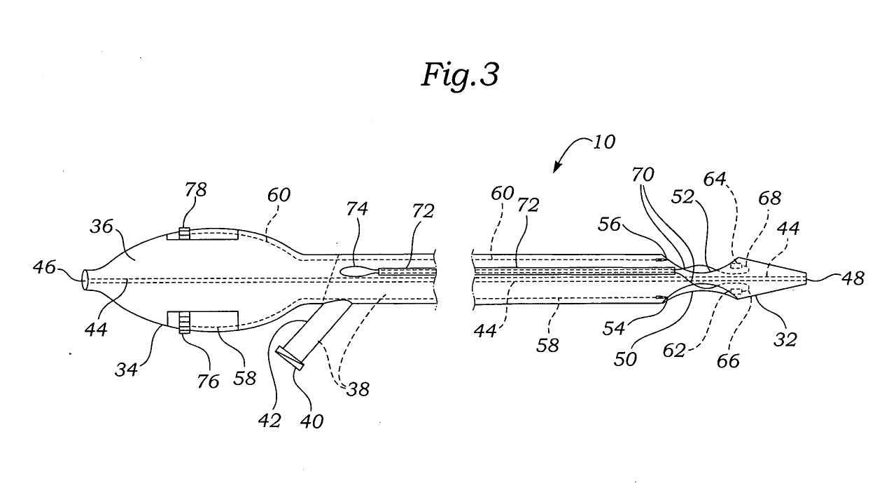

FIG. 3 depicts the treatment catheter 10 in greater detail. A vacuum lumen 38 passes through the catheter body 30 from the distal end 32 to the proximal end 34, where it terminates in a vacuum attachment adaptor 40 positioned on a y-connector 42. A guidewire lumen 44 also passes through the catheter body 30, terminating in a proximal guidewire opening 46 at the proximal end 30 and in a distal guidewire opening 48 at the distal end 32.

The distal end 32 has a first vacuum recess 50 and a second vacuum recess 52, each of which leads to the vacuum lumen 38. The first and second vacuum recesses 50, 52 have first and second needles 54, 56, respectively. The first and second needles 54, 56 are secured to first and second needle drivers 58, 60 respectively, which can longitudinally advance and retract the needles across their respective vacuum recesses. On the needle opposite sides in each vacuum recess are first and second needle catchers 62, 64, which are configured to be speared by their respective needles 54, 56 and drawn back with the needles when the needles are retracted. The first and second needle catchers 62, 64 are secured to first and second suture ends 66, 68. In the embodiment depicted, the first and second suture ends 66, 68 are opposing ends of a common suture thread 70. In the particular embodiment depicted, the common suture thread 70 runs longitudinally in a small sleeve 72 along the outside of the catheter elongated body 30, forming a loop 74 near the proximal end. The positioning of the common suture thread 70 in the small sleeve 72 on the outside of the catheter body 30 prevents tangling of the suture 70 when the treatment catheter 10 is removed toward the end of the procedure. Additionally, by causing the suture thread to pass on the outside of the catheter around a side surface 75 of the catheter body 30 which passes between the first and second vacuum recesses, 50, 52, the suture can be applied to tissue and the catheter withdrawn without the suture thread 70 becoming tangled or wrapped around the structures forming the catheter distal end 32.

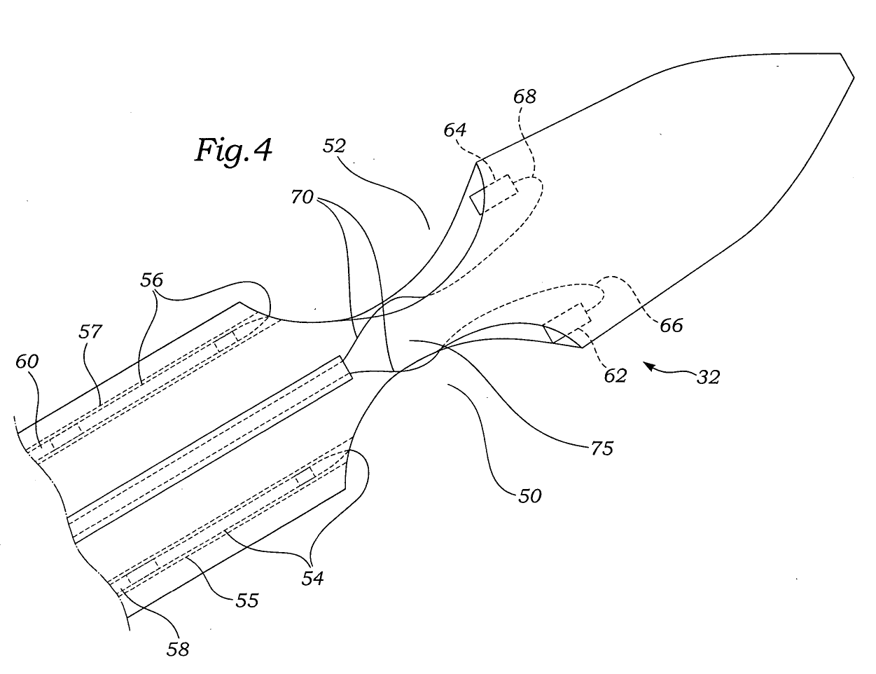

FIG. 4 depicts a close-up view of the distal end 32. In the embodiment of FIG. 4, the vacuum recesses 50, 52 are generally crescent-shaped in profile and are circumferentially spaced apart about the elongated catheter body 30. In the particular embodiment of FIG. 1, the spacing is 180 degrees, so that the vacuum recesses 50, 52 are on opposite sides of the elongated body 30. The needles 54, 56 are slidingly positioned within guide lumens 55, 57 that align the needles with their respective needle catchers 62, 64.

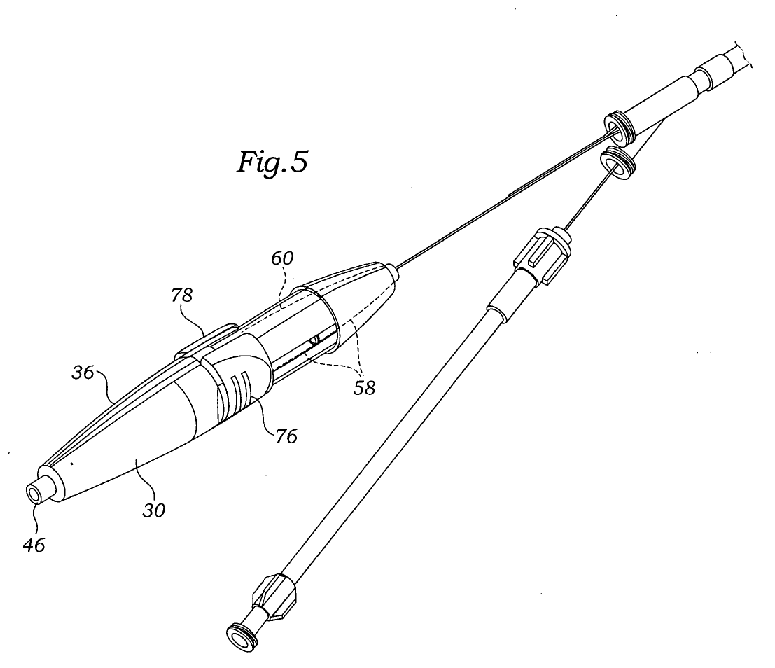

FIG. 5 depicts a close-up view of the proximal end 30. The handle 36 comprises a first sliding knob 76 which, when advanced distally or retracted proximally, causes, via the first needle driver 58, the first needle 54 to be advanced or retracted. On the opposite side of the handle is a second sliding knob 78 which, when advanced distally or retracted proximally, causes, via the second needle driver 60, the second needle 56 to be advanced or retracted.

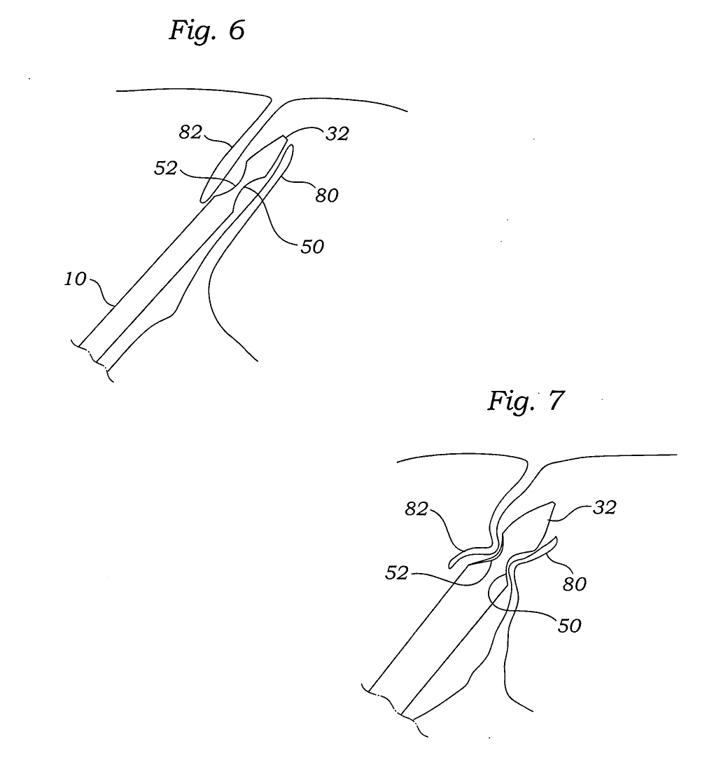

FIG. 6 depicts the treatment catheter 10 with the distal end 32 advanced into the PFO 14 between adjacent tissue, namely the septum primum 80 and the septum secundum 82. Note that in most persons the septum primum 80 will have healed permanently to the septum secundum 82, thereby permanently closing the foramen ovale. In a patient having a PFO, however, the septum primum 80 will not have healed in the closed position, and instead acts a flap that only partially occludes blood flow between the right and left atria 20, 22. In FIG. 6, the distal end 32 is positioned within the PFO 14 with the first vacuum recess 50 positioned adjacent the septum primum 80, and the second vacuum recess 52 positioned adjacent the septum secundum 82. In FIG. 7, vacuum is applied (via the vacuum attachment adaptor 40 and vacuum lumen 38) to the vacuum recesses 50, 52. The resulting suction draws the septum primum 80 into the first vacuum recess 50, and the septum secundum 82 into the second vacuum recess 52.

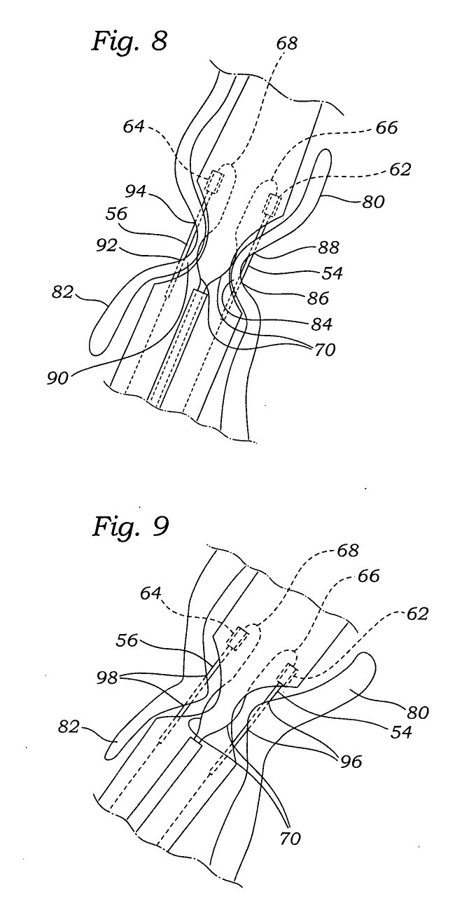

In FIG. 8, the first and second needles 54, 56 are driven (via actuation by a user advancing the sliding knobs 76, 78 depicted in FIG. 5) forward and through the tissue of the septum primum 80 and septum secundum 82 and into the needle catchers 62, 64, respectively. In the particular embodiment depicted, the first needle 54 is driven through a suction-created fold or crease 84 in the septum primum 80, thereby creating two needle holes 86, 88 in the septum primum 80. Similarly, the second needle 56 is driven through a suction-created fold or crease 90 in the septum secundum 82, thereby creating two needle holes 92, 94 in the septum secundum 82. Note, however, that if the septal tissue is thicker and/or the respective vacuum recess is smaller, the first and/or second needles 54, 56 will each form only a single needle hole 96, 98 when driven through the tissue, as depicted in FIG. 9.

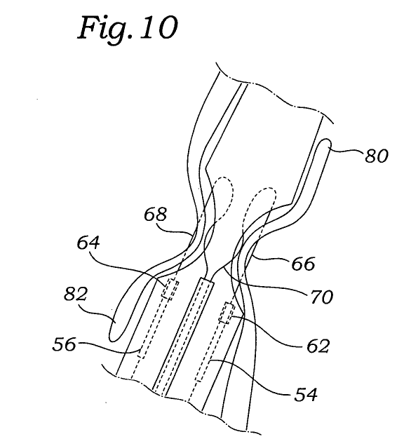

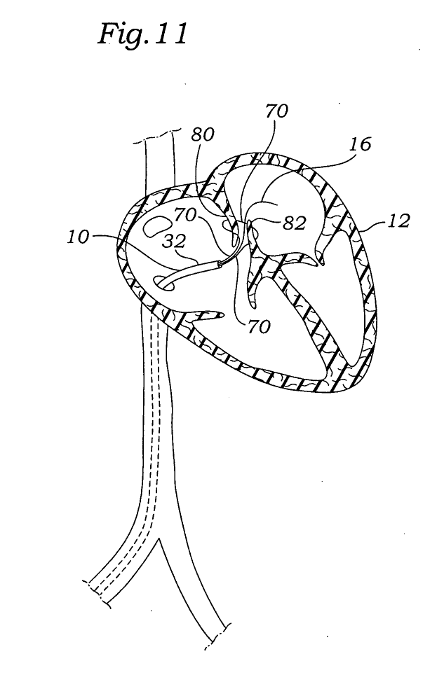

FIG. 10 depicts the needles 54, 56 retracted, thereby pulling the needle catchers 62, 64 and suture ends 66, 68 back through the septum primum 80 and septum secundum 82. With the suture thread 70 thus passing through both the septum primum 80 and the septum secundum 82, the treatment catheter 10 is withdrawn, as depicted in FIG. 11. Note the suture thread 70 playing out as the treatment catheter 10 is withdrawn. Note that the guidewire 16, if used in the particular procedure, will typically remain in position while the treatment catheter 10 is withdrawn.

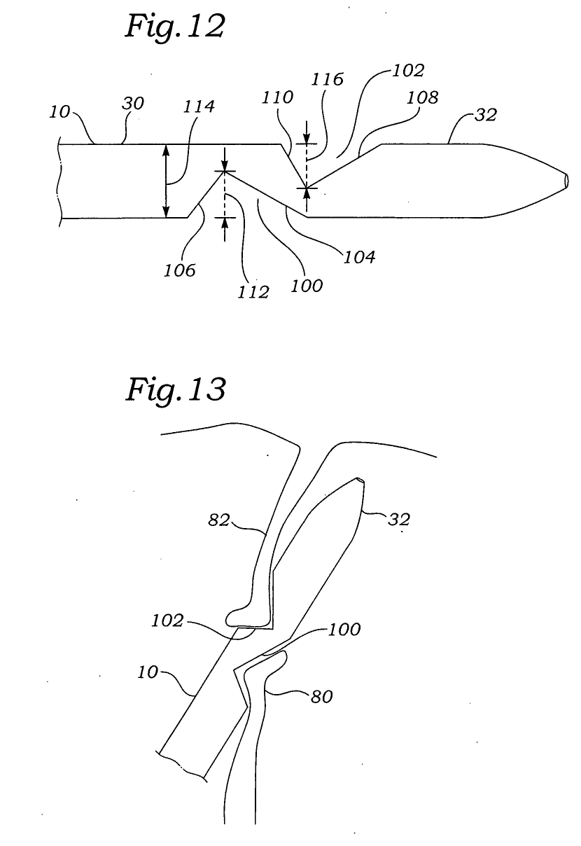

FIG. 12 depicts another embodiment, with the treatment catheter distal end 32 having first and second vacuum recesses 100, 102 that are generally v-shaped in profile. The distal portion 104 of the first vacuum recess 100, which forms the distal part of the v-shape, is substantially longer than the proximal portion 106, with a ratio of about 3:1 proximal length to distal length. The same is true for the second vacuum recess 102, where the distal portion 108 of the second vacuum recess 102 is substantially longer than the proximal portion 110. Note that the respective sizes of the distal portion to the proximal portion of one or both vacuum recesses can vary, depending on the particular application. For example, the distal portion of one or both vacuum recesses could be shorter than the proximal portion, or the distal and proximal portions of one or both ports could be generally of the same size, etc.

It is further noted that a variety of shapes could be used for the vacuum recesses, including U-shaped, three-sided rectangular, etc. Also, the vacuum recesses do not have to be mirror images of each other. For example, a particular shape may be particularly suited to grasping one tissue piece, such as the septum primum, while another shape may be better suited to grasping another tissue piece, such as the septum secundum.

In FIG. 12, the first and second vacuum recesses 100, 102 are longitudinally displaced along the elongate body 30, with the second vacuum recess 102 distal of the first vacuum recess. This longitudinal displacement can allow the vacuum recesses to have greater depth, which can increase the ability of the vacuum recess to hold particular tissue. If the vacuum recesses are longitudinally aligned, the depth of each port is limited by the thickness of the treatment catheter and by the depth of the opposite vacuum recess. For example, for a device having a diameter of 5 mm with vacuum recesses opposite and longitudinally aligned, the maximum combined depth of the two ports must be well under 5 mm (accounting for some structure therebetween to hold the catheter together). For such aligned ports that each have the same depth, their maximum depth in a 5 mm thick treatment catheter would be less than 50% of the catheter diameter, or under 2.5 mm depth each. However, if the ports are longitudinally displaced, as in the embodiment of FIG. 12, the depths of the vacuum recesses are limited only by the diameter of the catheter. As depicted in FIG. 12, the first vacuum recess 100 has a depth 112 that is about 70% of the catheter diameter 114. The depth 116 of the second vacuum recess 102 is also about 70% of the catheter diameter 114.

Depending on the application, longitudinal displacement of the vacuum recesses can also improve the ability of the device to grasp tissue. For example, where the septum primus 80 and septum secundum 82 have little or no overlap, as depicted in FIG. 13, the use of longitudinally off-set vacuum recesses 100, 102 can improve the ability of the user to capture the respective tissue with the device.

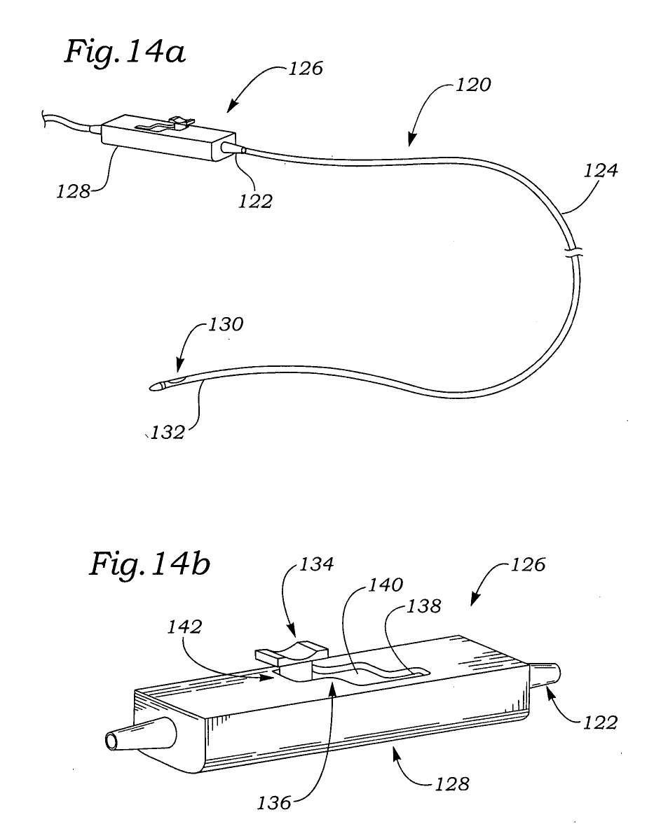

FIGS. 14-17 show various illustrations of a fastener catheter of the present invention. As shown in FIG. 14a, the fastener catheter 120 comprises a fastener catheter body 124 having a fastener catheter handle 126 attached at the proximal end 128 and a fastening tip 130 at the distal end 132. The fastener catheter 120 may be manufactured in a variety of shapes, sizes, lengths, widths, and biologically-compatible materials as desired.

FIG. 14b shows a more detailed illustration of a preferred fastener catheter handle 126 of the present invention. As shown, the fastener catheter handle 126 comprises a fastener handle body 128 having a fastener body receiver 122 attached thereto. The fastener body connector 122 is capable of receiving and coupling to the fastener catheter body 124 (FIG. 14a). A fastener actuator 134 may be positioned within a fastener actuator recess 136 formed on the fastener handle body 128. The fastener actuator 134 positioned within the fastener actuator recess 136 may be capable of being positioned in three distinct locations. For example, in a non-actuated condition, the fastener actuator 134 may be located in a first position 138. Thereafter, the user may partially actuate the fastener catheter 120 by positioning the fastener actuator 134 in a second position 140, thereby deploying a fastening device (not shown) from the fastener catheter 120. The user may then fully actuate the fastener catheter 120 by moving the fastener actuator 134 to a third position 142 within the fastener actuator recess 136, thereby actuating a cutting member (discussed below) located on or proximate to the fastening tip 130.

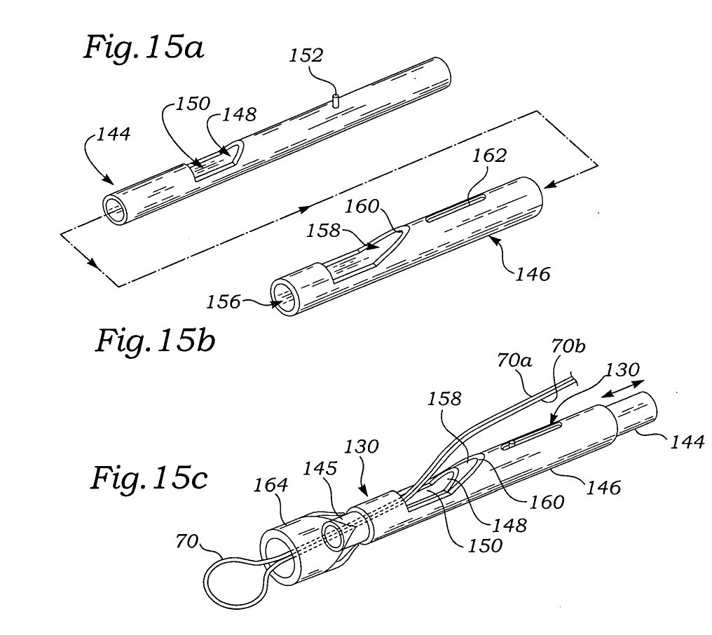

FIGS. 15a-15b illustrate, in exploded fashion, pieces of fastening tip 130, which is shown assembled in FIG. 15c. An inner body 144 includes a suture recess 150 formed in the side thereof, which in turn is in communication with an internal fastener lumen 148. Inner body 144 also includes a pin 152 extending radially outward therefrom. Sleeve 146 comprises an axial deployment lumen 156 of sufficient diameter to receive inner body 144 therein. Sleeve 146 also comprises a cutting recess 158 formed in an axial side thereof and a cutting member 160 which, in the embodiment depicted, is on a proximal edge of cutting recess 158. Slot 162 extends parallel to the axis of the deployment lumen 156 and may extend radially through to fastener lumen 148. Pin recess 162 receives pin 152 in sliding relation.

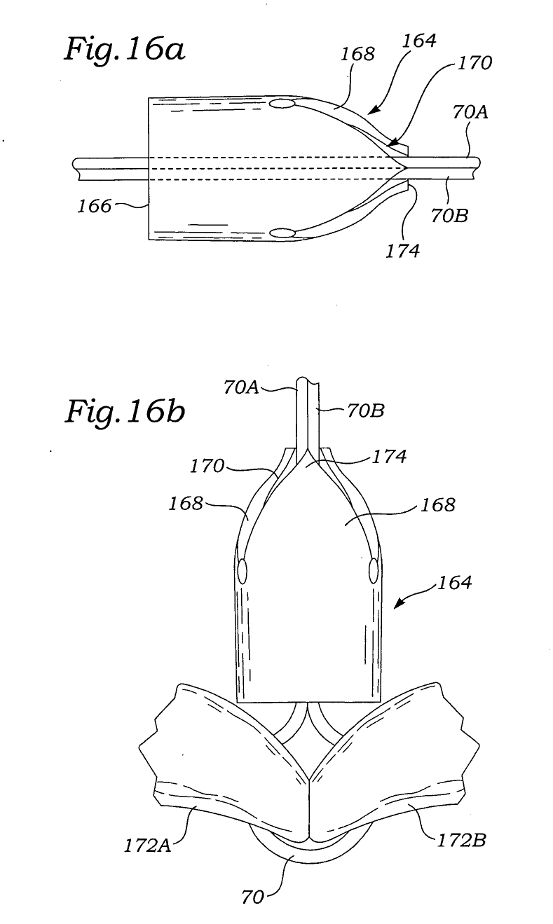

FIGS. 16a and 16b illustrate a fastener 164 of the present invention. Fastener 164 may be manufactured from a variety of materials including, for example, Nickel-Titanium alloys, shape-memory alloys, stainless steel, titanium, various plastics, and other biologically-compatible materials. Fastener 164 has an internal attachment lumen 166 extending axially therethrough and one or more engagement member(s) 168 formed on a proximal end thereof. Between the engagement members is defined engagement aperture 170 which is in communication with attachment lumen 166. Attachment lumen 166 and engagement aperture 170 are sized to receive a first suture lead 70A and a second suture lead 70B therein. Prior to deployment, engagement member(s) 168 are deflected radially away from the axis of the fastener 164 such that engagement aperture 170 has a relative large first diameter sufficient to permit suture leads 70A and 70B to slide therethrough. Upon deployment, i.e. after the suture leads 70A and 70B have been retracted, engagement members 168 are deflected or permitted to spring back toward the central axis of the fastener 164 such that the engagement aperture 170 assumes a second smaller diameter compressing and securing suture leads 70A and 70B in place. Depending on the particular embodiment, including the materials from which a particular fastener is made, the engagement member(s) 168 may tend to spring toward a natural position at the axis of fastener 164. FIG. 16b shows the fastener 164 in the deployed configuration in which a suture loop 70 has passed through two tissue portions 172A, 172B and suture leads 70A, 70B are secured in fastener 164. Each engagement member(s) 168 may further include a pointed tip 174 which, when the engagement member(s) are in the deployed position, engages and further restricts movement of the suture leads 70A, 70B. Other fasteners may also be used without departing from the scope of the invention. Examples of some fasteners are set forth in co-pending U.S. patent application Ser. No. 10/389,721, filed May 14, 2003 and entitled “Mitral Valve Repair System and Method of Use,” the contents of which are incorporated herein by reference in their entirety.

An operational fastening tip 130 with fastener 164 attached thereto and ready for deployment can be seen in FIG. 15c. Inner body 144 has been placed inside sleeve 146 such that suture recess 150 is in alignment with cutting recess 158. Pin 152 is in slidable communication with slot 162 thereby permitting relative linear motion, but preventing relative rotational motion, between inner body 144 and sleeve 146. Fastener 136 has been placed on the end of the fastening tip 126 by deflecting the engagement members 168 radially outward until they can be placed around the outer circumference of a distal end 145 the inner body 144. Accordingly, the fastener is secured to the inner body distal end 145 by means of the frictional engagement between the engagement members 168 and the outer surface of inner body 144. Suture 70 extends from the fastener 164, with suture leads 70A and 70B extending through the fastener lumen 166, through engagement aperture 170, exit the side of inner body 144 through suture recess 150, and exit the side of sleeve 146 through cutting recess 158.

Deployment of the fastener is a two step process. Once suture 170 has been secured through one or more tissue segments, the fastener tip 126 is coaxed toward the tissue and the suture leads 70A and 70B are pulled away from the tissue until the suture 70 is sufficiently cinched around the target tissue. Sleeve 146 is then held in place adjacent the tissue while the inner body 144 is pulled axially away. This causes sleeve 146 to push (i.e. slide) fastener 164 off the inner body distal end 145. When fastener 164 has been completely removed from inner body distal end 145, the fastener engagement members 168 spring axially inward thereby reducing the diameter of engagement aperture 170 and securing suture leads 70A and 70B. The second deployment step, cutting suture leads 70A and 70B, is accomplished when the inner body 144 is pulled sufficiently through sleeve 146 that the suture leads 70A, 70B are pinched between the distal edge of suture recess 150 and cutting member 160 and ultimately cut by cutting member 160.

Remote deployment of fastener 164 is accomplished by attaching inner body 144 to fastener actuator 134, and attaching sleeve 146 to the fastener catheter handle 126. Thus, axial movement of the fastener actuator 134 relative to the handle 126 causes similar relative movement between inner body 144 and sleeve 146. For example, in the non-actuated position 138 (see FIG. 14b) the distal end 145 of inner body 144 will extend from sleeve 146 a sufficient distance to hold fastener 164 thereon. In the second position 140 the inner body distal end 145 will have been withdrawn into sleeve 146 a sufficient distance to deploy the fastener 164, and in the third position 142 the inner body 144 will have been withdrawn a sufficient distance to cut the suture leads 70A and 70B. Note that other fastener catheters may also be used without departing from the scope of the invention. Examples of some fastener catheters are set forth in co-pending U.S. patent application Ser. No. ______, filed concurrently herewith and entitled, “System, Apparatus, and Method for Fastening Tissue,” the contents of which are expressly incorporated herein by reference in their entirety.

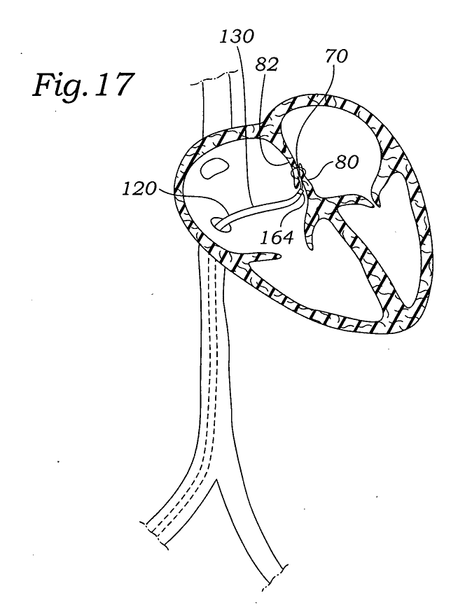

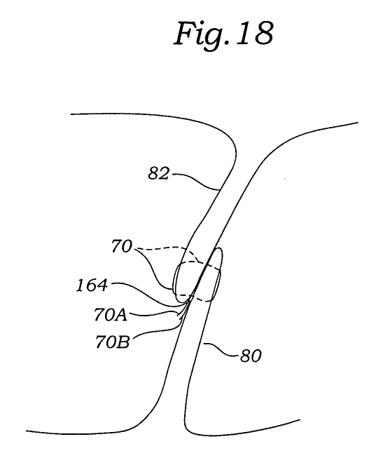

Further details on using the fastener catheter 120 for treating a PFO are depicted in FIG. 17. With the treatment catheter 10 withdrawn, the user can advance the fastener catheter 120 into the vicinity of the PFO. The fastener catheter 120 will advance along the suture 70 and, if present, along the guidewire. The user can initially tighten the suture 70 with the fastener catheter 120, thereby determining whether the suture 70 is properly positioned to close the PFO by drawing the septum primum 80 to the septum secundum 82. The advancement of the fastener catheter 120, combined with the user holding (and possibly pulling on) the suture portions 70A, 70B, cause the suture 70 to tighten. If the suture 70 was deployed properly in the desired tissue, the septum primum 80 will close against the septum secundum and seal the PFO as the suture 70 is tightened. The user can verify the effectiveness of the closure by monitoring various patient functions. For example, the user may confirm the closure of the PFO by monitoring blood flow using radiopaque dyes combined with fluoroscopy. If the user is dissatisfied with the closure when the suture is initially tightened, the user can remove the suture entirely from the patient and being the procedure again to try to achieve a better positioning of suture to the PFO. If the user is satisfied with the closure, the fastener catheter 120 can deploy the suture clip 164 that holds the suture 70 in its tightened position, thereby permanently closing the foramen ovale. The user can then use the fastener catheter 120 to cut the suture 70 in relatively close proximity to the suture clip 164. The user then withdraws the fastener catheter 120 from the patient, leaving the suture 70 and suture clip 164 in place to hold the septum primum 80 and septum secundum 82 together to close the foramen ovale, as depicted in FIG. 18. The guidewire, if present, will then be removed.

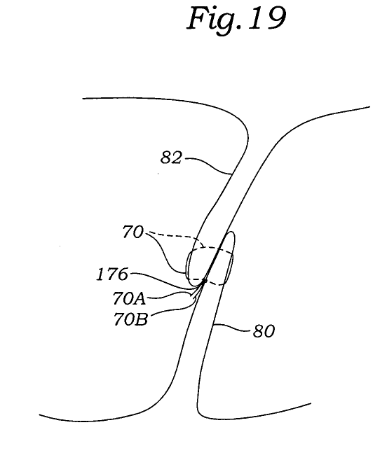

Another embodiment for securing the suture 70 is to tie the suture into a knot 176, as depicted in FIG. 19. The knot 176 could be tied in the suture within the patient's body, such as may be the case where the knot is tied in the suture adjacent the operational site (i.e., adjacent the septum primum 80 and septum secundum 82). The knot 176 may also be tied in the extraneous suture material some distance from the operational site, or even outside the patient's body. For example, the knot may be tied loosely and/or as slip knot in the suture some distance from the operational site, and then the knot can be advanced to the operational site. Advancing the knot to the operational site may involve the use of surgical knot pushers and methods such as depicted and described in co-pending patent application Ser. No. 09/797,964, filed on Feb. 6, 2001 and entitled “Surgical Knot Pushing Device and Method of Use,” now issued as U.S. Pat. No. 6,860,890, the contents of which are incorporated herein by reference in their entirety. As the knot is advanced, the suture at the operational site is tightened until the septal tissue is held in proximity. Depending on the particular application, one or more knots can be used to secure the suture and close the PFO. Once the final knot has been applied and/or advanced to the operational site, the extraneous suture material is trimmed.

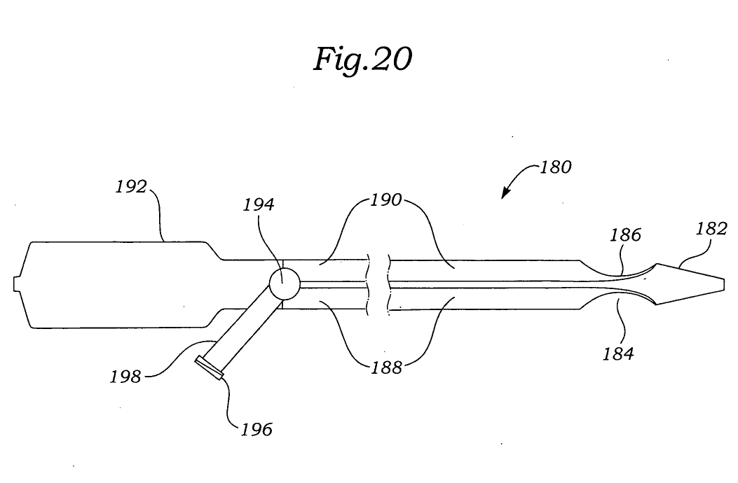

In the embodiment depicted in FIGS. 3-10, the vacuum applied to either of the vacuum recesses 50, 52 could not be controlled independently of the vacuum applied to the other vacuum recess. Depending on the particular embodiment and application, however, the vacuum recesses may have individually controllable vacuums. For example, in the particular treatment catheter 180 depicted in FIG. 20, the distal end 182 has first and second vacuum recesses 184, 186 fed by separate first and second vacuum lumens 188, 190 that run to the proximal end 192 of the treatment catheter. At the proximal end, a vacuum control valve 194 independently controls the application of vacuum (via the vacuum attachment adaptor 196 positioned on the y-connector 198) to each vacuum lumen 188, 190, thereby effectuating independent application of vacuum to each vacuum recess 184, 186. Although only a single control valve 194 is depicted in FIG. 20, other arrangements for providing independent control are within the scope of the invention, such as having a separate control valve for each vacuum lumen. Note that the treatment catheter 180 of FIG. 20 may include a guidewire lumen, and will also have the ability to suture the tissue via elements such as slidable needles, needle catchers, suture, etc., although these elements are not depicted in FIG. 20. Using a device with independent vacuum control such as that depicted in FIG. 20, a user could simultaneously (or near-simultaneously) grasp the septum primum and septum secundum by simultaneously applying vacuum to both vacuum lumens 188, 190 and both vacuum recesses 184, 186. The user could then simultaneously pass the needles and suture through the septum primum and septum secundum, or pass the needles and suture sequentially, i.e., first through the septum primum and then through the septum secundum, or first through the septum secundum and then through the septum primum. A user could also grasp the septum primum and septum secundum sequentially by sequentially applying vacuum sequentially to vacuum lumens 188, 190 and vacuum recesses 184, 186. For example, a user could apply vacuum only to the first vacuum lumen 188, and thereby only to the first vacuum recess 184, and thereby grasp only the septum primum. The user could then apply the needle and suture to the septum primum, discontinue the vacuum to the first vacuum recess 184 and thereby release the septum primum, and then apply vacuum to the second vacuum lumen 190 and second vacuum recess 186, and thereby grasp the septum secundum, followed by application of the needle and suture to the septum secundum. The user could also use a device such as that of FIG. 20 to apply vacuum initially to just the first vacuum recess 184, and thereby grasp the septum primum, and then apply vacuum to the second vacuum recess 186 while maintaining vacuum to the first vacuum recess 184, so that the septum secundum is grasped while the septum primum is still held by the vacuum. Such an application may be helpful where the septum primum and septum secundum are positioned apart, and a user can thus use the device to grasp the septum primum and move it toward the septum secundum, or to grasp the septum secundum and move it toward the septum primum.

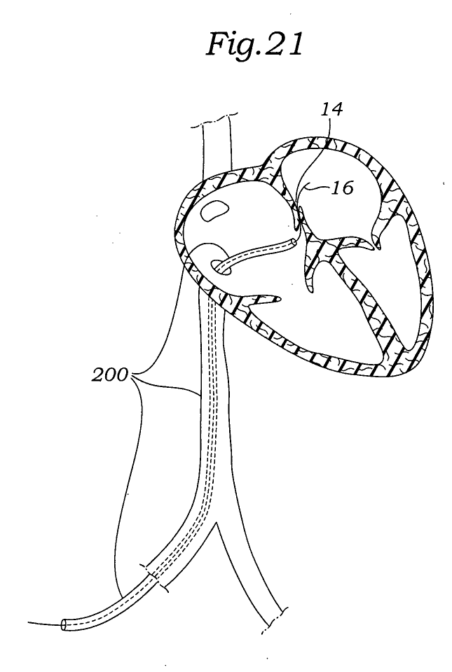

In the embodiment depicted in FIGS. 1, 11, and 17, the treatment and fastener catheters were advanced and positioned using a guidewire, but without a guide catheter. Depending on the particular application, however, including such factors as the flexibility or steerability of the treatment and/or fastener catheters, a guide catheter may be used, typically in combination with the guide wire. As depicted in FIG. 21, the guide catheter 200 can be advanced over the guidewire 16 to a position adjacent the PFO 14. The treatment and fastener catheters can then be advanced and withdrawn through the guide catheter 200.

The above designs provide for smooth and predictable deployment of the suture as the treatment catheter is removed from the patient. For example, in the embodiment depicted in FIG. 3, opposing ends 66, 68 of the suture 70 came out of each vacuum recess 50, 52, passed into the external sleeve 72, and terminated in a loop 74 near the treatment catheter proximal end 34. This structure allows the suture 70 to freely come out of the catheter, and prevents the suture 70 from becoming wrapped around various parts of the catheter. Other designs can also provide for smooth deployment of the suture.

While the invention has been described with reference to particular embodiments, it will be understood that various changes and additional variations may be made and equivalents may be substituted for elements thereof without departing from the scope of the invention or the inventive concept thereof. For example, while the invention is specifically discussed in application with repair of septal defects such as PFOs, it has applicability in other areas where it is desired to repair tissue. In addition, many modifications may be made to adapt a particular situation or device to the teachings of the invention without departing from the essential scope thereof. Therefore, it is intended that the invention not be limited to the particular embodiments disclosed herein, but that the invention will include all embodiments falling within the scope of the appended claims.

What is claimed is:

1. A method of treating a patent foramen ovale, the method comprising:
providing a catheter device having a proximal end, a distal end, at least one vacuum recess near the distal end, and a connector near the distal end;
advancing the catheter through a patient's circulatory system to position the distal end within the patent foramen ovale;
applying a vacuum to the first vacuum recess to stabilize a first tissue portion adjacent the patent foramen ovale; and
securing the connector to the first tissue portion.

2. The method of claim 1, further comprising:
securing the connector to a second tissue portion adjacent the patent foramen ovale; and
drawing the first tissue portion toward the second tissue portion.

3. The method of claim 2, wherein the connector comprises a suture.

4. The method of claim 2, wherein the catheter device comprises a second vacuum recess, and further comprising:
applying a vacuum to the second vacuum recess to stabilize the second tissue portion adjacent the patent foramen ovale.

5. The method of claim 4, wherein applying the vacuum to the first vacuum recess occurs simultaneously with applying the vacuum to the second vacuum recess.

6. The method of claim 4, wherein applying the vacuum to the first vacuum recess occurs after applying the vacuum to the second vacuum recess.

7. An apparatus for treating tissue, the apparatus comprising:
an elongated body having a proximal end and a distal end;
a first vacuum recess near the distal end; and
a second vacuum recess near the distal end, the second vacuum recess circumferentially displaced about and longitudinally displaced along the elongated body from the first vacuum recess.

8. The apparatus of claim 7, wherein the first vacuum recess comprises a generally v-shaped profiled opening in the elongated body.

9. The apparatus of claim 7, wherein the elongated body has a diameter, and the first vacuum recess has a depth greater than 50% of the elongated body diameter.

10. The apparatus of claim 7, further comprising:
a first needle adjacent the first vacuum recess, the first needle slidingly received in a first needle lumen, wherein the first needle is configured to be slidingly advanced across the first vacuum recess.

11. The apparatus of claim 10, further comprising:
a second needle adjacent the second vacuum recess, the second needle slidingly received in a second needle lumen, wherein the second needle is configured to be slidingly advanced across the second vacuum recess.

12. The apparatus of claim 11, further comprising:
a first needle catcher, the first needle catcher secured to a first end of a suture line; and
a second needle catcher, the second needle catcher secured to a second end of the suture line.

13. The apparatus of claim 7, wherein the first vacuum recess comprises a generally v-shaped opening in the elongated body.

14. The apparatus of claim 13, wherein the first vacuum recess v-shaped opening is generally asymmetrical from a side view.

15. The apparatus of claim 14, wherein the first vacuum recess generally v-shaped opening comprises a first v leg and a second v leg, wherein the first v leg is longer than the second v leg.

16. A device for treating tissue, comprising:
an elongated body having a proximal end and a distal end;
a first vacuum recess near the distal end;
a second vacuum recess near the distal end, wherein the elongated body distal end includes at least one side comprising the surface passing between the first vacuum recess and the second vacuum recess;
a first tissue connector positioned adjacent or within the first vacuum recess;
a second tissue connector positioned adjacent or within the first vacuum recess; and
a suture line connecting the first tissue connector to the second tissue connector, the suture line passing on the outside of the elongated body on the side comprising the surface passing between the first vacuum recess and the second vacuum recess.

17. The device of claim 16, further comprising:
a sleeve on the elongated body, wherein the suture line passes within the sleeve.

18. The device of claim 17, wherein the sleeve is positioned on the outside surface of the elongated body.

19. The device of claim 17, wherein the suture line enters the sleeve at a first opening, forms a loop, and exits the sleeve at the first opening.

20. The device of claim 16, wherein the first and second tissue connectors are positioned within the first and second vacuum recesses, respectively, and the suture line exits the first vacuum recess, passes over the outside of the elongated body on the side defining the surface which passes between the first vacuum recess and the second vacuum recess, and enters the second vacuum recess.
